(19) United States
(12) Patent Application Publication
(10) Pub. No.: US 2022/0167953 A1
(43) Pub. Date: Jun. 2, 2022

(54) Microbial Diagnostic Sampling Swab

(71) Applicants: Javad Parvizi, Gladwyne, PA (US); Karan Goswami, Philadelphia, PA (US)

(72) Inventors: Javad Parvizi, Gladwyne, PA (US); Karan Goswami, Philadelphia, PA (US)

(21) Appl. No.: 17/521,501

(22) Filed: Nov. 8, 2021

Related U.S. Application Data
(60) Provisional application No. 63/111,261, filed on Nov. 9, 2020.

(51) Int. Cl. A61B 10/02; C12Q 1/6806

(52) U.S. Cl. CPC A61B 10/02; C12Q 1/6806

(57) ABSTRACT

A method of using a Defined Swab for genetic identification of microbes, said method comprising contacting said swab with a target to absorb a sample of said target; extracting said sample from said swab; and microbiologically testing said sample to identify said microbe.

### REFERENCE TO RELATED APPLICATION

This application is based on U.S. Provisional Application No. 63/111,261, filed Nov. 9, 2020, which is hereby incorporated by reference in its entirety, including its appendices.

### FIELD OF INVENTION

The present application relates, generally, to swabs for obtaining samples for diagnostic testing and identification of microbial organisms, and, more specifically, to the use of swabs for microbial molecular testing—via polymerase chain reaction, next generation sequencing, metagenomic sequencing and other molecular methodologies—for the purpose of infection diagnosis or identification of microbially contaminated surfaces.

### BACKGROUND

Surgical site infection (SSI) is a devastating complication. Across surgical fields, SSI causes up to an 11-fold risk in mortality. Although most patients do recover with appropriate treatment, 77% of mortality in patients with an SSI is attributable to the infection. Infection is of particular concern in patients undergoing surgery with an implanted device. In orthopedic surgery, periprosthetic joint infection (PJI) after total joint arthroplasty is a relatively uncommon complication, but remains extremely challenging to diagnose and treat, and is associated with increased morbidity and mortality for the patient as well as a burden on the healthcare system. PJI is the leading cause of revision after total knee arthroplasty, and is a major cause for revision after arthroplasty of the hip, shoulder, elbow, and ankle.

PJI and other implant-related infections are often challenging due to the formation of a biofilm on the surface of the implant. These infections occur when organisms adhere to the surface, and proliferate over the course of weeks to form micro-colonies. Organisms embedded in a biofilm are poorly recognized by the immune system, and persist to cause chronic infection in the host. The slow growth of organisms in a biofilm also create challenges for treatment, especially with growth-dependent antimicrobial agents.

Whether treating an infection associated with native tissue or on the surface of an implant, diagnosis and successful treatment of SSI and PJI requires an accurate identification of the causative organism. Identification of the organism(s) responsible for infection of a patient requires collection of specimens, which can include bodily fluids, tissue, and the sampling of surfaces, such as the nasal cavity, mouth, or infected tissue with a swab. Plating of these specimens in growth media has long been the standard for growth and identification of microorganisms. While tissue and fluid culture remain integral to diagnosis and organism identification, the rate of PJI in which cultures yield no results is between 7% and 50%, and, thus, it is clear that these methods are not sufficient.

In recent years the use of more advanced techniques, such as the amplification and measure of microbial genetic material through the use of genomic testing (e.g. polymerase chain reaction or next-generation sequencing). The techniques offer greater speed and sensitivity than traditional techniques. Although these techniques enable the detection of microbes at very low levels, the accuracy of these tests depends greatly on the quality of the sample collected.

There is a need for innovation and for rethinking the current practice. Molecular testing including PCR and next-generation sequencing (NGS) have shown promising results in this context. A swab such as the one developed in this study could represent a cost-efficient method of reliably collecting microbial DNA of pathogenic organisms.

To achieve high accuracy with genomic testing, there is a need for a sampling swab having not only the ability to absorb biotic materials, including any pathogens, within the fabric, but also the ability to release those materials into fluid for further processing and testing, a process known as extraction. The present invention fulfills this need among others.

### SUMMARY

The following presents a simplified summary of the invention in order to provide a basic understanding of some aspects of the invention. This summary is not an extensive overview of the invention. It is not intended to identify key/critical elements of the invention or to delineate the scope of the invention. Its sole purpose is to present some concepts of the invention in a simplified form as a prelude to the more detailed description that is presented later.

It has been discovered that the sampling swab described in U.S. Pat. No. 8,745,806, herein incorporated by reference, when used with a double-layered woven polyester fabric and tip of sufficient size can increase diagnostic yield of microbial genomic testing for identification of contaminated surfaces and causative organisms of infected patients. In addition, Applicants' data suggests the material weave pattern and device described confer optimized SARS-CoV-2 testing yield from patients, as detailed below.

Accordingly, in one embodiment, the invention relates to a new use for a sampling swab to collect microbiological samples for molecular diagnostic testing is provided. The swab possesses a large sampling surface as well as high absorption/adsorption and extraction properties to increase yield of various testing modalities. A quick-release mechanism allows easy transfer of swab head to collection device while reducing inadvertent contamination risk.

### BRIEF DESCRIPTION OF FIGURES

FIG. 1 shows Ct values of various swabs for planktonic S. aureus and E. coli.

FIG. 2 shows Ct values of various swabs for biofilm S. aureus and E. coli.

FIG. 3 shows Ct values of prior art swabs following 24-hour hold.

FIG. 4 shows Ct values of various swabs using buffer solution for S. aureus.

FIG. 5 shows Ct values of various swabs using buffer solution for E. coli.

FIG. 6 shows Ct values of various swabs using buffer solution compared to dry 36-hour hold.

FIG. 7 shows microscopic views of polyester fabric with (A) Sheath Pull Tight over Fixed Member, and (b) Standard Woven Pattern

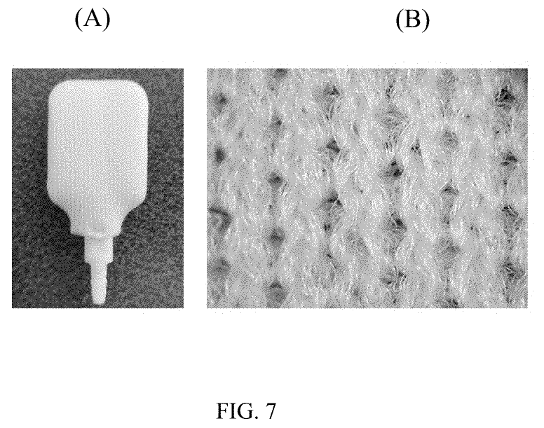

FIG. 8 shows a standard swab's deficiency in surface area to collect specimens and its inability to scrap against tissue or surfaces to lift the DNA and RNA compared to the swab of the present invention.

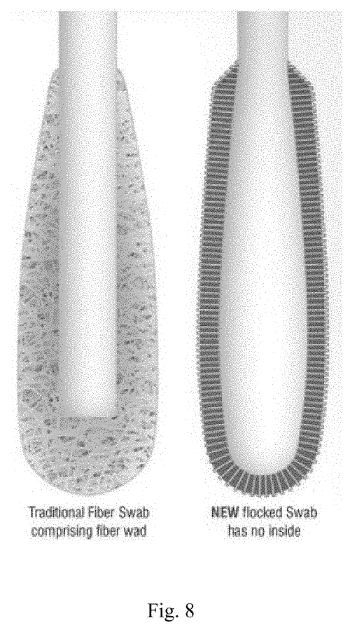

### DETAILED DESCRIPTION

In the following paragraphs, the present invention will be described in detail by way of example with reference to the attached drawings. Throughout this description, the preferred embodiment and examples shown should be considered as exemplars, rather than as limitations on the present invention. As used herein, the “present invention” refers to any one of the embodiments of the invention described herein, and any equivalents. Furthermore, reference to various feature(s) of the “present invention” throughout this document does not mean that all claimed embodiments or methods must include the referenced feature(s).

The SurfCHECK Cleaning Validation Swab, Foamtec International WCC as disclosed in U.S. Pat. No. 8,745,806 is currently marketed as a tool to validate cleaning processes, specifically within the pharmaceutical industry. As used herein, the term “Defined Swab” refers to the swab disclosed in in U.S. Pat. No. 8,745,806) It is offered as a multi-use tool, including multiple swab heads that can be used to swab a surface, and then be ejected into a container by way of a push button mechanism on the handle. This mechanism reduces the risk of cross contamination between samples or from the swab becoming contaminated by the user. It is used to verify cleaning through the use of several chemical analyses, including total organic carbon (TOC), high performance liquid chromatography (HPLC), ion-mobility spectrometry (IMS), and ultraviolet-visible spectroscopy (UV-Vis).

Applicants have discovered that in addition to cleaning validation and chemical analyses, this swab has a unique ability to absorb and release microbial particles that is superior to many swabs currently used for this application.

Real-time polymerase chain reaction (RT-qPCR) is a common microbial molecular diagnostic technique. This technique allows quantification of microbes within a sample. This is achieved by amplifying microbial genetic material in cycles. As genetic material is amplified, a fluorescent signal intensifies until it crosses a particular threshold, signifying a positive result. Fewer cycles required to cross threshold corresponds to higher quantities of microbes in the original sample.

This swab, with a 15×25 mm tip and double-woven polyester fabric, was gamma-irradiated to sterilize and compared to four sampling swabs currently marketed. When adjusting for the difference in surface area between swabs, this swab required approximately 30% fewer cycles in detecting Staphylococcus aureus, a common gram-positive pathogen, to the next highest-performing swab and 40% fewer cycles in detecting Escherichia coli, a common gram-negative pathogen. Although a swab having a 15×25 mm tip and double-woven polyester fabric provided sufficient results, other embodiments exist, for example, a swab with an area of at least 250 mm2, or at least 300 mm2, or at least 350 mm2, or at least 375 mm2, or at least 400 mm2, or at least 450 mm2 or at least 500 mm2.

The swab, as described in the previous paragraph, was tested against the Clinical & Laboratory Standards Institute (CLSI) M40-A2 Standard for Quality Control of Microbiological Transport Systems. This includes testing the ability to detect several bacterial species representing the entire diverse spectrum, after several time points from collection, including 0, 24, and 48 hours. This swab met all the requirements described in this standard.

The swab is not only useful for detecting bacteria but can be used for other microbial pathogens. The swab was tested in its ability to detect the novel SARS-CoV-2 virus. Samples collected using the swab were found to exceed the threshold of detection even after a five-fold dilution, even after a 36-hApplicants' hold at room temperature. Oropharyngeal swabbing with this swab demonstrated a detectable lower limit that was 32 times lower samples collected from nasopharyngeal swabs. With the addition of a buffer solution to preserve genetic material, samples collected using this swab were able to detect viral concentrations as low 0.1 viral copies per microliter.

The preferred form of the swab for the described application is as a single-use device that comes pre-loaded with a single tip encased in two layers of woven polyester fabric. In one embodiment, the woven polyester fabric comprising a double layer of woven polyester fabric utilizing a standard warp and weft patterns, such as a plain weave, twill or leno type of patterns that provide open channels while remaining strong and durable. The swab is sealed in medical grade peel pack packaging and sterilized to reduce the risk of contamination.

### Examples

In these non-limiting examples, Applicants demonstrate that the swab material disclosed herein is highly effective in its absorption and release of S. aureus and E. coli. Specifically, through comparative materials testing, Applicants identify a swab effective in detecting these organisms both as planktonic bacteria and in a biofilm, throughout an extended holding period, and in various buffer solutions. After identification of an optimal swab material, Applicants assessed the final swab using the CLSI M40-AD standard for microbiological culture.

Methods

Comparative testing of materials for absorption and extraction of planktonic Gram-positive and Gram-negative organisms on PCR

The first experiment aimed to compare materials and weave patterns for optimal absorption and extraction of planktonic organisms. A panel of 11 candidate gauze and swab weave patterns were included in the experiment. All materials were spiked with a standardized inoculum of Gram-positive (Staphylococcus aureus), or Gram-negative (Escherichia coli) planktonic organisms. Following a holding period, each candidate sample was extracted and tested via PCR as described below.

Comparative testing of materials for biofilm collection, absorption and extraction of Gram-positive and Gram-negative organisms on PCR

Next, Applicants assessed a high-performing subset of the first experiment against the same organisms in biofilm form. To this end, biofilms consisting of monocultures of either S. aureus (ATCC 25923) or E. coli (ATCC 11303) were grown on the surface of 12 mm Polycarbonate transwell inserts. Once the biofilms were grown, each insert was removed from its original growth well and transferred to a new well containing a single gauze testing material (approximately 1 cm²; each sample was previously weighed for subsequent normalization). Each biofilm was removed via debridement (vortex with uniform force applied). The samples were then transferred for testing of absorptive, biofilm removal, and extraction efficiencies.

Comparative Testing of Materials for Absorption and Extraction of Gram-Positive and Gram-Negative Organisms on PCR, Following 24 Hours of Simulated Transport Specimen Hold

Monocultures consisting of either S. aureus (ATCC 25923) or E. coli (ATCC 11303) were grown in 12-well plates. After 24 hours each gauze sample was briefly submerged in culture. The samples were then transferred into tubes for 24 hours at 25° C. to mimic the time of transport. Samples were then assayed to test for absorptive and nucleic acid extraction efficiencies.

Comparative Buffer Testing

To assess the buffer transport utility, biofilms consisting of monocultures of either S. aureus (ATCC 25923) or E. coli (ATCC 11303) were grown on the surface of 12 mm Polycarbonate transwell inserts. After biofilms were grown, each insert was removed from its original growth well and transferred to a new well containing a single gauze testing material (approximately 1 cm²; each sample was previously weighed for subsequent normalization). Each biofilm was removed via debridement (vortex with uniform force applied). The samples were then transferred into different buffers for testing of absorptive, biofilm removal and nucleic acid extraction efficiencies.

Assessment of Candidate Swab to the CLSI M40 A2 Standard for Microbiological Culture

The test procedures employed for determining bacterial viability performance were based upon the quality control methods described in Clinical Laboratory Standards Institute (CLSI) M40-A2. Test organisms utilized in this study were those specifically listed in M40-A2 for establishing performance claims and quality control of swab transport systems. These include a representative panel of aerobes, anaerobes and fastidious bacteria. Bacterial viability studies were performed on the candidate swab, as well as another widely used commercially available swab, at room temperature. Swabs accompanying each transport system were inoculated in triplicate with 100 μl of specific concentrations of organism suspension. Swabs were then placed in their respective transport medium tubes and were held for 0 hrs, 24 hrs or 48 hrs. At the appropriate time intervals, each swab was processed according to the Roll-Plate or Swab Elution Method.

PCR Testing

In each of the preceding experiments, PCR testing was performed per standardized protocols at CLIA-certified MicroGenDx Laboratories (Lubbock, Tex.). Samples were mechanically lysed and extracted using a Qiagen TissueLyser in combination with the Roche High Pure PCR Template Preparation kit. Each sample was tested for 16S abundance to verify species, and to test for possible contaminates obtained during the procedure. All experimental conditions were repeated in triplicate.

Results

Comparative Testing of Materials for Absorption and Extraction of Planktonic Gram-Positive and Gram-Negative Organisms on PCR

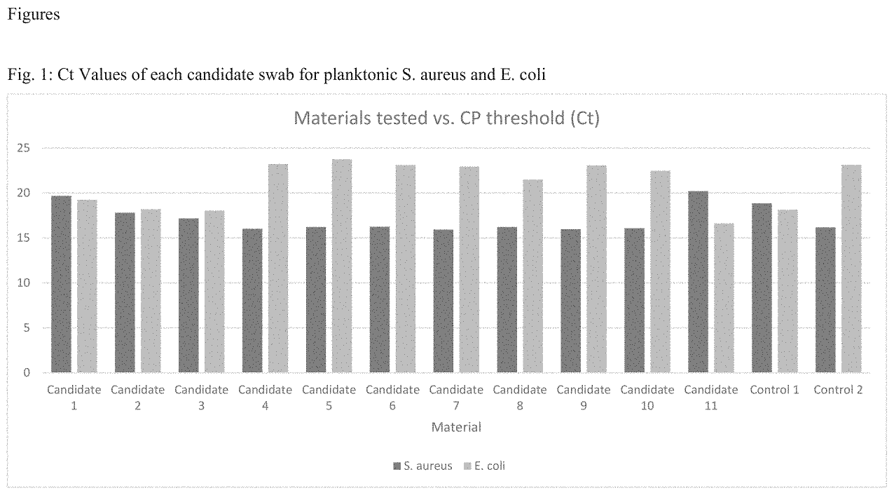

Ct thresholds were calculated for 11 candidate materials and two controls (FIG. 1). The average Ct across all materials for S. aureus was 17.05, while the average Ct across all materials for E. coli was 21.38. Six candidates (highlighted in Table 1) demonstrated consistent absorption and extraction results for planktonic forms of both S. aureus and E. coli (Table 1).

Comparative testing of materials for biofilm collection, absorption and extraction of Gram-positive and Gram-negative organisms on PCR

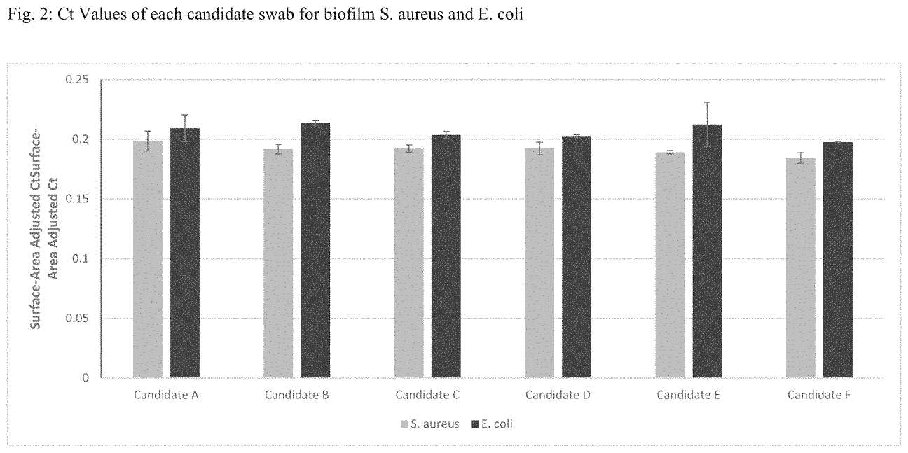

The six remaining candidates that demonstrated optimal results for planktonic bacteria were further analyzed for absorption and extraction of organisms in a biofilm. Ct thresholds were calculated after adjusting for surface area (FIG. 2). No significant differences between candidate for S. aureus biofilm, but significant differences were noted between candidates for E. coli biofilm (Table 3) as well as between S. aureus and E. coli for three of the candidates (Table 4).

Comparative Testing of Materials for Absorption and Extraction of Gram-Positive and Gram-Negative Organisms on PCR, Following 24 Hours of Simulated Transport Specimen Hold

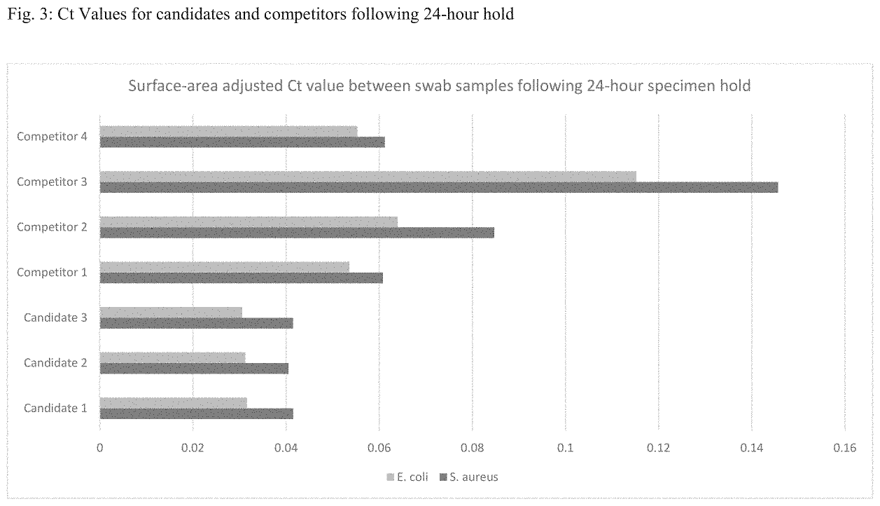

Three candidates were selected to assess performance following 24-hApplicants' hold, compared to four commercially available competitors. FIG. 3 displays Ct values for S. aureus and E. coli following 24-hApplicants' hold. Candidates 2 and 3 demonstrated significantly lower Ct values compared to competitor for S. aureus, while all three candidates demonstrated lower Ct values for E. coli.

Comparative Buffer Testing

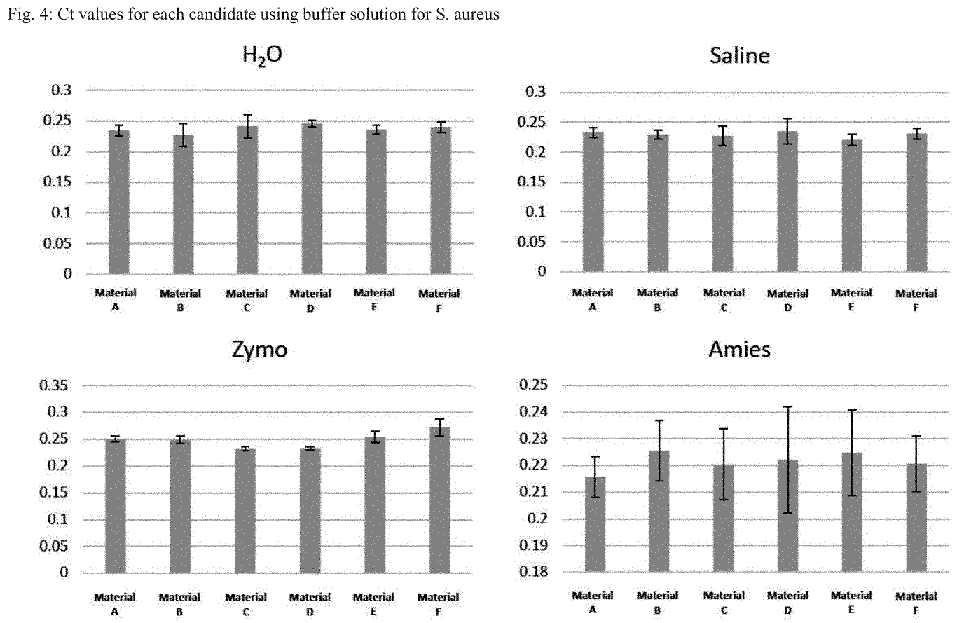

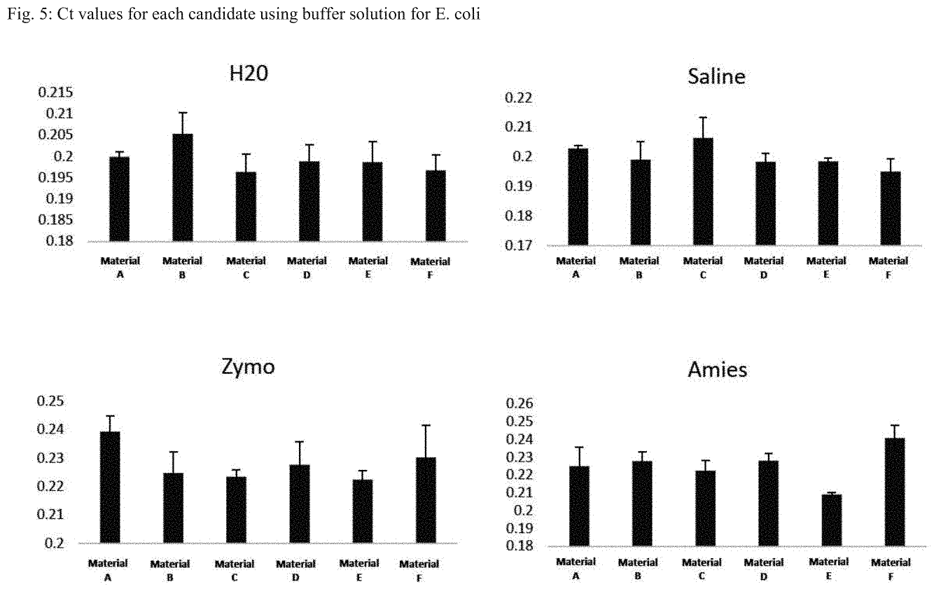

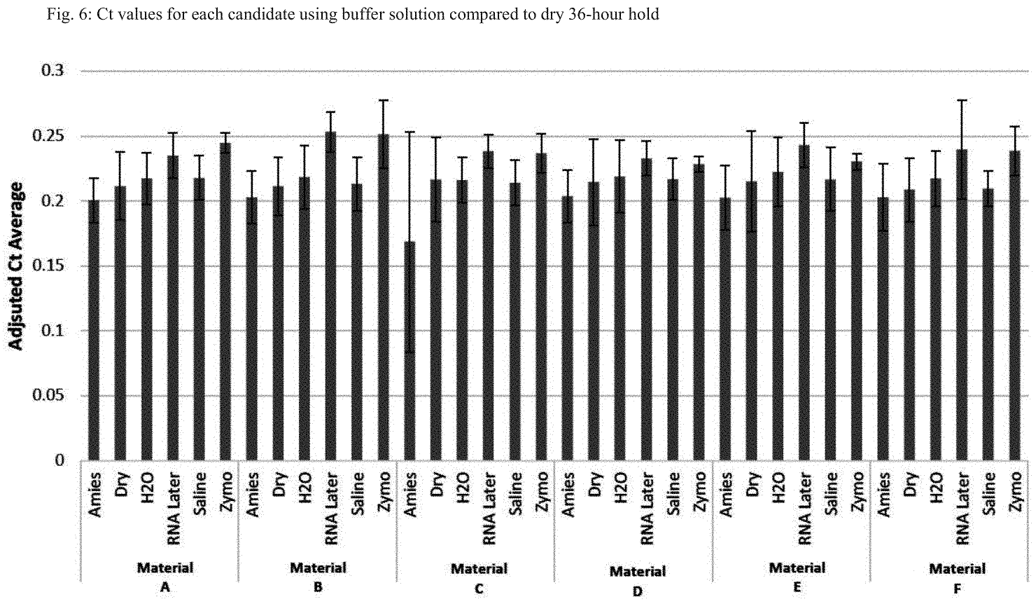

Normalized surface-area adjusted Ct values for the six candidates above were calculated in four buffer solutions for performance with S. aureus (FIG. 4) and E. coli (FIG. 5). Each was also compared with the same material in a “dry” control condition (FIG. 6). Minimal, if any, differences were noted between the buffers tested vs. dry 36-hApplicants' hold conditions.

Assessment of Candidate Swab to the CLSI M40 A2 Standard for Microbiological Culture

Two candidate swabs were compared to a single competitor swab according the CLSI M40-A2 Standard. Results are reported in Table 5. Both candidates met all requirements per the standard.

Discussion

Through a series of in vitro experiments, Applicants have found a specific polyester weave pattern that performs the best among all tested fabrics in absorbing, storing, and releasing DNA of a given pathogen. The material performed well in the collection of S. aureus and E. coli in both planktonic form and in biofilm, which is a crucial aspect of detecting an organism in implant-related infections. The improvements in quality sample collection by use of this swab may aid in reducing the rate of culture-negative PJI. This may have further effect in improving outcomes associated with PJI as cases of culture-negative PJI only have a 70% treatment success rate.

Furthermore, the swab performed very well even after an extended 24 hours of simulated transport. Molecular testing often occurs in centralized laboratories, rather than individual hospitals. It is therefore necessary for a swab system to maintain organism viability while the specimen is in route to the facility for analysis. Additionally, after a 36-hApplicants' dry hold, the swab demonstrated similar results to those preserved in a series of buffer solutions. While the addition of a buffer solution can stabilize a sample for a prolonged period, availability of molecular testing supplies has become limited in the current epidemic, including supplies used for nucleic acid storage and stabilization.′ The swab developed demonstrates the ability to provide reliable results following transport without the dependence on such buffer solutions.

While S. aureus and E. coli were chosen to test candidate materials with both Gram-positive and Gram-negative organisms, it is possible that another material may have performed better with other organisms chosen from the same categories. Although several candidates were chosen with potential for high performance, it is possible that a material not tested in this study would demonstrate superior results. From the results of this study, it is not known how the surface being swabbed affects performance or if certain swabs perform better on certain sampling surfaces. However, the swab demonstrated its efficacy against the array of organisms shown in the results of CLSI M40-A2 testing.

While molecular testing techniques offer the potential for significant improvement in the identification of causative organisms, the performance of these techniques hinge on the quality of samples provided. Use of a superior sampling swab, as demonstrated in Applicants' results, may even further improve the success of these novel techniques.

Having thus described a few particular embodiments of the invention, various alterations, modifications, and improvements will readily occur to those skilled in the art. Such alterations, modifications, and improvements as are made obvious by this disclosure are intended to be part of this description though not expressly stated herein, and are intended to be within the spirit and scope of the invention. Accordingly, the foregoing description is by way of example only, and not limiting. The invention is limited only as defined in the following claims and equivalents thereto.

Tables

TABLE 1

| | CP threshold (Ct) Values | |
|---|---|---|
| Material | S. aureus | E. coli |
| Candidate 1 | 19.68 | 19.25 |
| Candidate 2 | 17.81 | 18.2 |
| Candidate 3 | 17.17 | 18.04 |
| Candidate 4 | 16.02 | 23.23 |
| Candidate 5 | 16.77 | 23.75 |
| Candidate 6 | 16.25 | 23.11 |
| Candidate 7 | 15.93 | 22.93 |
| Ca.ndidate 8 | 16.22 | 21.49 |
| Candidate 9 | 15.98 | 23.07 |
| Candidate 10 | 16.08 | 22.48 |
| Candidate 11 | 20.21 | 16.62 |
| Control 1 | 18.85 | 18.14 |
| Control 2 | 16.18 | 23.13 |

TABLE 2

| | S. aureus | | E. coli | |
|---|---|---|---|---|
| | | SA-adjusted | | SA-adjusted |
| Material | CP threshold | Ct value | CP threshold | Ct value |
| Candidate A | 19.85 | 0.1985 | 20.92 | 0.2092 |
| Candidate B | 19.18 | 0.1918 | 21.37 | 0.2137 |
| Candidate C | 19.22 | 0.1922 | 20.37 | 0.2037 |
| Candidate D | 19.23 | 0.1923 | 20.27 | 0.2027 |
| Candidate E | 18.91 | 0.1891 | 21.24 | 0.2124 |
| Candidate F | 18.42 | 0.1842 | 19.76 | 0.1976 |

SA—surface area

TABLE 4
P-values of comparison between S. aureus and
E. coli for each candidate

| | | P-value (one- |
|---|---|---|
| Material | P-value | tailed) |
| Candidate A | 0.37878 | 0.18939 |
| Candidate B | 0.009283 | 0.004642 |
| Candidate C | 0.006465 | 0.003232 |
| Candidate D | 0.052233 | 0.026116 |
| Candidate E | 0.146111 | 0.073055 |
| Candidate F | 0.034463 | 0.017231 |

TABLE 5
CLSI M40A-2 Standard Testing Results

| | | | Quantitative (Swab Elution) CFU/ml | | | Qualitative (Roll Plate) CFU | | | |
|---|---|---|---|---|---|---|---|---|---|
| | | | | | M40-A2 | | | | M40-A2 |
| Organism | Swab | Temperature | 0 hr | 48 hr | Compliance | 0 hr | 24 hr | 48 hr | Compliance |
| Cutibacterium acnes | Candidate 1 | Room Temp | 4.80E+06 | 1.49E+07 | ✓ | 312 | | 35 | ✓ |
| ATCC 6919 | Candidate 2 | | 5.30E+06 | 1.61E+07 | ✓ | 174 | | 14 | ✓ |
| | Competitor | | 6.37E+06 | 1.52E+07 | ✓ | 242 | | 59 | ✓ |
| Neisseria gonorrhoeae | Candidate 1 | RoomTemp | 5.60E+06 | 1.62E+07 | ✓ | 149 | 76 | | ✓ |
| ATCC 43069 | Candidate 2 | | 6.18E+06 | 1.58E+07 | ✓ | 248 | 97 | | ✓ |
| | Competitor | | 6.38E+06 | 1.48E+07 | ✓ | 197 | 96 | | ✓ |
| Fusobacterium nucleatum | Candidate 1 | Room Temp | 2.20E+07 | 1.95E+07 | ✓ | 142 | | 47 | ✓ |
| ATCC 25586 | Candidate 2 | | 2.18E+07 | 1.99E+07 | ✓ | 241 | | 21 | ✓ |
| | Competitor | | 2.20E+07 | 2.03E+07 | ✓ | 230 | | 96 | ✓ |
| Haemophilus influenzae | Candidate 1 | Room Temp | 1.49E+07 | 1.60E+07 | ✓ | 269 | | 70 | ✓ |
| ATCC 10211 | Candidate 2 | | 7.21E+05 | 3.00E+07 | ✓ | 320 | | 82 | ✓ |
| | Competitor | | 7.68E+06 | 2.41E+07 | ✓ | 264 | | 75 | ✓ |
| Streptococcus pneumoniae | Candidate 1 | Room Temp | 1.76E+07 | 2.85E+07 | ✓ | 170 | | 13 | ✓ |
| ATCC 6305 | Candidate 2 | | 1.37E+07 | 1.90E+07 | ✓ | 182 | | 28 | ✓ |
| | Competitor | | 3.75E+06 | 5.44E+06 | ✓ | 134 | | 11 | ✓ |
| Streptococcus pyogenes | Candidate 1 | Room Temp | 1.71E+07 | 1.42E+07 | ✓ | 284 | | 76 | ✓ |
| ATCC 19615 | Candidate 2 | | 6.35E+06 | 1.83E+07 | ✓ | 178 | | 44 | ✓ |
| | Competitor | | 1.88E+07 | 1.09E+07 | ✓ | 282 | | 92 | ✓ |
| Prevotella melaninogenica | Candidate 1 | Room Temp | 1.58E+06 | 2.31E+07 | ✓ | 264 | | 64 | ✓ |
| ATCC 25845 | Candidate 2 | | 1.85E+07 | 8.50E+06 | ✓ | 134 | | 61 | ✓ |
| | Competitor | | 1.56E+07 | 1.35E+07 | ✓ | 178 | | 31 | ✓ |
| Bacteroides fragilis | Candidate 1 | Room Temp | 8.95E+06 | 2.02E+07 | ✓ | 210 | | 38 | ✓ |
| ATCC 25285 | Candidate 2 | | 9.72E+06 | 4.80E+06 | ✓ | 196 | | 97 | ✓ |
| | Competitor | | 2.46E+06 | 1.81E+07 | ✓ | 160 | | 83 | ✓ |
| Peptostreptococcus | Candidate 1 | Room Temp | 2.08E+07 | 1.51E+07 | ✓ | 226 | | 44 | ✓ |
| anaerobius | Candidate 2 | | 5.15E+06 | 9.14E+06 | ✓ | 140 | | 37 | ✓ |
| ATCC 27337 | Competitor | | 9.46E+06 | 1.75E+07 | ✓ | 280 | | 63 | ✓ |
| Pseudomonas aeruginosa | Candidate 1 | Room Temp | | | ✓ | | | | ✓ |
| ATCC | Candidate 2 | | | | ✓ | | | | ✓ |
| | Competitor | | | | ✓ | | | | ✓ |

What is claimed is:

1. A method of using a Defined Swab for genetic identification of microbes, said method comprising:
contacting said swab with a target to absorb a sample of said target;
extracting said sample from said swab; and
microbiologically testing said sample to identify said microbe.

2. The method of using a swab of claim 1, wherein said target is a biological surface.

3. The method of using a swab of claim 2, wherein said target is a biofilm.

4. The method of using a swab of claim 3, wherein said biofilm is on an animal.

5. The method of using a swab of claim 4, wherein said animal is a human.

6. The method of using a swab of claim 5, wherein said biofilm is on a surface of an implant in said human.

7. The method of using a swab of claim 1, wherein said extraction comprises debriding.

8. The method of using a swab of claim 1, wherein said microbiologically testing comprises testing for DNA/RNA of said microbe.

9. The method of using a swab of claim 1, wherein said testing comprises mechanically lysing said sample and extracting using the Qiagen TissueLyser in combination with the Roche High Pure PCR Template Preparation kit.

10. The method of using a swab of claim 1, wherein said swab comprises a polyester knitted fabric.

11. The method of using a swab of claim 10, wherein said swab comprises a swab tip comprising a double layer of woven polyester fabric having standard warp and weft patterns.

12. The method of using a swab of claim 1, wherein said swab comprises a swab tip an area of at least 350 mm2,

13. The method of using a swab of claim 12, wherein said swab tip has two flat sides having a combined area of at least 350 mm2.

14. The method of using a swab of claim 13, wherein said combined area is at least 375 mm2.

15. The method of using a swab of claim 14, wherein said combined area is at least 400 mm2.

16. The method of using a swab of claim 12, wherein said swab tip has two flat sides and approximate dimensions of 5-20 mm W×25-30 mm H×0.5-0.6 mm D.

17. The method of using a swab of claim 1, wherein said swab is sealed in a medical grade sterile package and sterilized.

18. The method of using a swab of claim 17, wherein said swab is sterilized by gamma-irradiation.
